(19) United States
(12) Patent Application Publication
(10) Pub. No.: US 2016/0287530 A1
(43) Pub. Date: Oct. 6, 2016

(54) METHODS FOR PREVENTION AND TREATMENT OF CARDIOMETABOLIC SYNDROME AND COMPOSITIONS USED THEREIN

(71) Applicant: OmniActive Health Technologies Limited, Mumbai (IN)

(72) Inventors: Jayant DESHPANDE, Charlottetown (CA); Abhijit BHATTACHARYA, Morristown, NJ (US); Vijaya JUTURU, Morristown, NJ (US)

(21) Appl. No.: 15/088,662

(22) Filed: Apr. 1, 2016

Related U.S. Application Data
(60) Provisional application No. 62/142,015, filed on Apr. 2, 2015.

(51) Int. Cl. A61K 31/047; G01N 33/92

(52) U.S. Cl. CPC A61K 31/047; G01N 33/92; G01N 2333/90283; G01N 2333/908; G01N 2333/90245; G01N 2333/70525; G01N 2333/485; G01N 2333/47; G01N 2333/90254; G01N 2800/32; G01N 2800/04

(57) ABSTRACT

Beta-cryptoxanthin compositions and methods are described for the management of cardiometabolic syndrome and associated risk factors, in a subject, in need thereof. Methods herein are directed to identifying such subject at risk of developing cardiometabolic syndrome and administering beta-cryptoxanthin composition to assess the condition of an organ. Compositions and methods herein can effectively reduce risk factors of cardiometabolic syndrome, such as hyperlipidemia, insulin resistance, obesity, diabetes, atherosclerosis and/or related cardiovascular disorders. Beta-cryptoxanthin compositions and methods herein can reduce body weight, body fat, glucose levels, and free fatty acids, when administered in effective amounts. The compositions and methods herein can also reduce oxidative stress on organs such as the eye and liver and/or reduce inflammatory and/or oxidative markers, when administered to subjects in need thereof.

### FIELD

Methods are described for the prevention and/or treatment of cardiometabolic syndrome and associated risk factors by administering beta-cryptoxanthin compositions in an effective amount to a subject in need thereof. More particularly, methods are described that relate to prevention and/or treatment of cardiometabolic syndrome such as hyperlipidemia, diabetes and other cardiovascular disorders by administering a composition comprising beta-cryptoxanthin as the active ingredient alone or in combination with other nutrients. The compositions herein help to improve cardiometabolic syndrome and manages associated risk factors such as body weight, body fats, lipid profile, blood glucose and the like, when administered in an effective amount to a subject in need thereof. The compositions herein also protect retina and liver by reducing oxidative stress and inflammatory markers, thus improving function of vital body organs, which are related to cardiometabolic syndrome. Beta-cryptoxanthin compositions described herein can reduce cardiometabolic stress in a subject in need thereof. The compositions herein are safe for human consumption and can be employed for management of cardiometabolic syndrome, when administered in an effective amount.

### BACKGROUND

Cardiometabolic syndrome is a disorder of energy intake, utilization and storage, diagnosed by a co-occurrence of three out of five of the following medical conditions: abdominal obesity, elevated blood pressure, elevated fasting plasma glucose, high serum triglycerides, and low high-density cholesterol (HDL) levels. The cardiometabolic syndrome is thus a combination of metabolic disorders, resulting into hyperlipidemia, impaired glucose tolerance, hypertension, oxidative stress and the tendency to develop fat around the abdomen.

Individuals with cardiometabolic syndrome are at high risk of developing heart failure and insulin resistance, thus affecting vital organs such as the eye, liver, kidney and nervous system. The body makes insulin to move glucose (sugar) into cells for use as energy. The prevalence of obesity is increasing at an alarming rate in developed and developing countries (Haslam and James, 2005). Obesity makes it more difficult for cells to respond to insulin. If the body cannot make enough insulin to override the resistance, the blood sugar level increases and diabetes can result. Although various risk factors such as age, high body mass index, smoking, stress, sedentary lifestyle, and postmenopausal status are identified, high fat diet is one of the most important risk factors leading to cardiometabolic syndrome.

Although during the last decades increasing scientific evidence has emerged that protective health effects can be obtained from diets that are rich in fruits, vegetables, legumes and whole grains, a significant number of the population still consumes junk food which is prevalent in high fat and refined carbohydrates. Such diet has been a critical factor blamed for obesity, diabetes, and a number of other cardiovascular diseases (Park et al., 2013). Rodents that are fed a high-fat diet develop visceral obesity, insulin resistance, hyperlipidemia, endothelial dysfunction, and hypertension (Roberts et al. 2001). Obesity is strongly associated with metabolic syndrome and many chronic diseases that result from an imbalance between energy intake and physical activity (Goran and Treuth 2001). It has significant health consequences such as diabetes, hypertension, cardiovascular disease and inflammatory disorders.

Excess energy intake and reduced energy expenditure promotes cardiometabolic dysfunction (Yang et al., 2009), oxidative stress and inflammatory pathologic factors that increase the secretion of interleukin-6 (IL-6), tumor necrosis factor-alpha (TNF-α), and C-reactive protein (CRP) (Wang et al., 2012). Increased adipose tissue plays an important role in the development of low-grade inflammation, which is characterized by cytokine production and stimulation of inflammatory cytokine signaling pathways (Hotamisligil and Erbay, 2008).

However, as a result of several epidemiologic studies and some clinical trials, it has been suggested that people with metabolic syndrome and obesity may benefit from intensive lifestyle modifications including dietary changes and adopting a physically more active lifestyle. (Pitsavos, Panagiotakos, et al., The Review of Diabetic Studies (2006) 3:118-126).

Carotenoids are known to play an important role in health and disease and the state of living human tissue(s) based on their antioxidant function and scavenging action on singlet molecular oxygen and peroxyl radicals (Stahl and Sies, 2003). Observational studies have proposed that dietary carotenoid intake or circulating carotenoid serum levels are associated with a reduced risk of mortality, cardiovascular disease, cancer, stroke, and other conditions (Krinsky and Johnson, 2005).

Beta-cryptoxanthin (BCX) is considered a provitamin A which is present in many fruits and vegetables and belongs to a family of carotenoids called xanthophylls. BCX is a long hydrocarbon chain and acts as an antioxidant that helps protect cells from free radical damage. By protecting cells from free radicals and by reducing free radical activities, it may prevent many dangerous diseases and conditions. Studies have found that BCX may reduce the risk of developing inflammatory disease. High intake of BCX has been shown to reduce the risk of rheumatoid arthritis, polyarthritis and other inflammatory diseases. BCX provides anti-aging benefits. As a powerful anti-oxidant, BCX helps keep skin cells healthy and young.

It is further observed that supplementation of highly concentrated BCX improves serum adipocytokine profiles in obese subjects consequently reduces risk of the progression of metabolic syndrome [Lipids Health Dis. 2012 May 14; 11:52]. BCX also facilitates lipid metabolism in muscle and reduced adipocyte proliferation and inflammatory response [Front Neurol. 2011 Nov. 23; 2:67].

Japanese Patent Application No. 2013173720A relates to carotenoid agents such as cryptoxanthin and/or an ester thereof for preventing retinopathy and is shown to exhibit superior prevention or amelioration effect on diabetic retinopathy.

Another Japanese Patent Application No. 2010273727 relates to an oral administration composition which exhibits the effect of reducing body fat, such as visceral fat, subcutaneous fat and the like, and neutral fat, and relates to a food, a pharmaceutical, and a feed having the composition; wherein the composition includes a carotenoid and a sphingolipid, or a carotenoid and a flavonoid and/or a derivative thereof. The cryptoxanthin and/or the sphingolipid and/or the flavonoid are derived from a citrus fruit and the citrus fruit is preferably Citrus unshiu.

Japanese Patent Application No. 2014162726 relates to the use of beta-cryptoxanthin and its derivatives to improve skin texture, to suppress chromatosis, in the retention of the skin, and in the evaluation as moisturizing agent, whitening agent, skin beautifier, and/or for preventing wrinkles. Thus beta-cryptoxanthin is explored for its cosmetic use in this patent application.

US Patent Application No. 20090258111A1 relates to a highly bioavailable cryptoxanthin composition (a food or drink or a feed) for oral administration wherein the amount of dietary fibers contained in the composition is 400 times by weight or less based on cryptoxanthin. The reference further relates to probable use of improved absorbability of cryptoxanthin into the living body for various effects such as its provitamin A activity and anti-oxidation action, carcinogenesis inhibitory action, osteogenesis accelerating action, and skin whitening action.

US Patent Application No. 20120053247 relates to a nutritional supplement including beta-cryptoxanthin, which may be used to maintain cardiovascular health by lowering blood pressure, preventing high, elevated blood pressure, and/or maintaining healthy blood pressure and reducing heart rate. The administration of beta-cryptoxanthin in combination with safflower oil is particularly effective at the amount of 0.1 mg and 20 mg per day.

W. P. Koh et al (Nutrition, Metabolism and Cardiovascular Diseases 21(9), 685-690, 2011) relates to a study of high plasma levels of beta-cryptoxanthin and lutein for decreasing the risk of acute myocardial infarction.

Sari Voutilainen et al (Am J Clin Nutr 83:1265-71, 2006) relates to the role of main dietary carotenoids such as lycopene, beta-carotene, alpha carotene, beta-cryptoxanthin, lutein, and zeaxanthin in the prevention of atherosclerosis.

Further Granado-Lorencio et al. (Nutr Metab Cardiovasc Dis. 2014 October; 24(10):1090-6) relates to the effect of beta-cryptoxanthin in combination with phytosterols (PS) on cardiovascular risk and bone turnover markers in post-menopausal women, wherein beta-cryptoxanthin improves the cholesterol-lowering effect of PS when supplied simultaneously.

### SUMMARY

Even though the literature above discusses the effect of beta-cryptoxanthin in cosmetic applications, body fat reduction, and diabetic retinopathy as well as for reducing risk of myocardial infarction, they do not relate to the effect of beta-cryptoxanthin compositions on management of metabolic syndrome and associated risk factors. Further none of the references relate to the evaluation of beta-cryptoxanthin compositions for their effect on basic cellular mechanism associated with diabetes, obesity and related cardiovascular risk factors in a subject, administered with high fat diet.

Cardiometabolic syndrome is an extremely complicated issue due to the fact that it encompasses a mesh of metabolic pathways (mainly glycemia and lipids) and involves several tissues (liver, fat, muscle, eye and others). For example, the liver is closely involved not only in regulation of glycemia and lipids but also in inflammation and hemostasis, which are main players in cardiometabolic syndrome.

Patients with diabetes/pre-diabetes, concomitant cardiovascular disease (CVD), and risk factors of hypertension, obesity, and/or dyslipidemia also have insulin resistance. The components of cardiometabolic syndrome also include abdominal obesity, diabetes, glucose intolerance, dyslipidemia, high blood pressure, and/or hyperuricemia. The association of diabetes and hypertension with ocular conditions such as retinopathy, cataract, and raised intraocular pressure (TOP) is well known. (Ref: Indian J Endocrinol Metab. 2012 March; 16(Supp 11): S6-S11, Ocular associations of metabolic syndrome, Rupali Chopra, Ashish Chander, and Jubbin J. Jacob) The prevalence of metabolic syndrome is rapidly increasing worldwide due for example to sedentary lifestyles. Its association with various ocular manifestations such as non-diabetic retinopathy, central retinal artery occlusion (CRAO), cataract, and primary open angle glaucoma suggests that an epidemic of metabolic syndrome can have far-fetched ocular consequences as well. Amelioration of metabolic syndrome may have a therapeutic role in preventing these ocular conditions.

Liver has an important role in cardiometabolic syndrome condition. The liver clears, metabolizes, detoxifies, and redistributes the absorbed content of food. Its role in established type 2 diabetes is well demonstrated, but increasing evidence implicates this organ in very early stages of prediabetes. One major finding of the last decade has been the recognition of a prevalence rate of 30% for hepatic steatosis in the general population, with an even higher prevalence in obese and elderly populations. The inflammatory state of the liver in prediabetic states is expected to impact on the cardiovascular system. There is indeed a well known interplay between insulin resistance, inflammation, obesity, and heart disease. Insulin-resistant individuals of normal weight have a 2.5-fold increase in risk for heart failure. The risk can be assessed by checking various biomarkers and the compositions having desirable effect on the biomarkers can be used effectively for managing cardometabolic syndrome. (Ref: Diabetes Metab Syndr Obes. 2013; 6: 379-388, Hepatic function and the cardiometabolic syndrome, Nicolas Wiernsperger)

Applicant has carried out rigorous experimentation and evaluation as reported herein exhibiting the use of beta-cryptoxanthin compositions in preventing and treating cardiometabolic syndrome by effectively reducing multiple risk factors such as hyperglycemia, hyperlipidemia and other cardiovascular disorders, in subjects fed with high fat diet. The effect of such compositions is described herein on biomarkers related to liver and eye. Methods for prevention and treatment of cardiometabolic syndrome as described herein are comprised of administering beta-cryptoxanthin compositions in an effective amount to a subject in need thereof and evaluating the effect on related oxidative and inflammatory biomarkers. The compositions as described herein are useful to improve cardiometabolic syndrome and manage associated risk factors such as body weight, body fats, lipid profile, blood glucose and the like. The compositions herein also protect retina by reducing oxidative stress. The compositions herein are safe for human consumption and can be employed for management of cardiometabolic syndrome, when administered in an effective amount.

In some embodiments, methods herein are directed to administering a beta-cryptoxanthin composition in an effective amount(s) to treat cardiometabolic syndrome in a subject and/or are directed to evaluating its beneficial effect on the management of cardiometabolic syndrome in a subject. As per the methods described herein, use of compositions is directed to prevention, treatment and improvement of associated risk factors, health conditions and vital body functions in a subject in need thereof, thus leading to overall management of cardiometabolic syndrome.

In one embodiment, methods herein are directed to evaluating the effect(s) of beta-cryptoxanthin composition on improvement of cardiometabolic health by administering to a subject in need thereof, an effective amount of a composition comprising beta-cryptoxanthin alone or in combination with other nutrients.

In one embodiment, methods described herein are comprised of administering beta-cryptoxanthin compositions for improvement of cardiometabolic health by increasing antioxidant activity and reduction in oxidative stress in retina and liver tissues.

In embodiment, methods for prevention and treatment of cardiometabolic syndrome are directed to administering beta-cryptoxanthin compositions for reducing oxidative stress and protecting the retina from neovascularization, when administered to a subject in need thereof, such as for example to a subject on a high fat diet.

In another embodiment, methods herein are directed to retarding the accumulation of lipofuscin pigment in retina and preventing the causes of retinal neovascularization, retinal vein occlusion, and/or neovascularization in peripheral retina by administering an effective amount of beta-cryptoxanthin either alone or in combination with other nutrient(s) including provitamin A to a subject in need thereof.

In one embodiment, methods described herein are directed to use of beta-cryptoxanthin compositions for prevention and treatment of cardiometabolic syndrome by reducing inflammatory and oxidative stress markers on associated vital body organs such as liver and eye.

In one embodiment, methods for improvement of cardiometabolic health are comprised of administering beta-cryptoxanthin compositions for management of a healthy lipid profile, reduction in body fat, visceral fat, and free fatty acid levels in the body.

In one embodiment, methods described herein relate to use of beta-cryptoxanthin compositions for management of metabolic syndrome, such as hyperglycemia, by the reduction in body glucose levels and/or the reduction in insulin resistance, in a subject fed with a high fat diet.

The beta-cryptoxanthin composition includes an active material present including beta-cryptoxanthin (BCX), which is extracted for example from paprika oleoresin by saponification followed by purification through column chromatography. Compositions herein are enriched with trans-beta-cryptoxanthin. In an embodiment, the extract is suspended in a suitable oil medium to obtain 5% oil suspension. In an embodiment, the suspension was evaluated in animal model described herein below. For human consumption, the compositions herein include final formulations into powders, granules, beadlets, and can be administered by oral solid dosage forms such as tablets, capsules.

In an embodiment, an effective amount herein relates to the amount of BCX present in the composition.

In an embodiment, a daily dose duration can range from at or about 3 months to at or about 2 years, or till the desired effect is achieved in a subject. It will be appreciated that there may be no fixed time period for the daily doses as it may be less or longer than such range. It will also be appreciated that the dose may be given continuously daily during this period or the administration can be stopped after obtaining a desired effect in a subject, and can also be restarted again as needed. It is appreciated that dose periods herein include the experiment durations or by general volunteer study period which can be extended to 12 months.

In one embodiment, methods described herein are comprised of administering beta-cryptoxanthin compositions in effective daily dose of at or about 0.1 to at or about 100 mg/kg body weight, for the treatment and/or management of cardiometabolic syndrome, to improve lipid profile, to reduce body weight, liver weight and and/or to reduce oxidative stress markers in the retina and/or liver.

In an embodiment, an effective daily dose includes a range of at or about 250 micrograms to at or about 30 mg/kg body weight.

In an embodiment, an effective daily dose includes a range of at or about 150 micrograms to at or about 20 mg/kg body weight.

In an embodiment, an effective daily dose includes a range of at or about 200 micrograms to at or about 10 mg/kg body weight.

In one embodiment, methods as described herein are directed to use of beta-cryptoxanthin compositions for reducing risk factors associated with cardiometabolic syndrome, such as for example obesity, diabetes, hypertension, and/or hyperlipidemia, and the like.

Methods and compositions described herein are useful for the treatment and/or management of cardiometabolic health in a subject in need thereof, such as for example a mammal fed with high fat diet, when administered in an effective amount.

In some embodiments, daily dose range can include about 250 micrograms to about 30 mg of beta-cryptoxanthin (BCX). In some embodiments, biological markers, genes, indicators, their regulated pathways, and the like include, but are not limited to data which have shown decreases in visceral fat, decreases in body weight, and decreases in liver weight. The methods as described herein are comprised of evaluation of use of beta-cryptoxanthin compositions in high fat diet (HFD) treated subjects, such as for example rats as an experimental model. Effects of BCX composition are evaluated on various health parameters associated with cardiometabolic syndrome such as total cholesterol, LDL cholesterol, triglycerides, glucose, insulin, leptin, adiponectin and free fatty acids.

In some embodiments, compositions herein include a beta-cryptoxanthin extract administered in the form of composition with other food grade excipients. Compositions herein can be in the form of oil suspensions, beadlets, spray dried powders, microcapsules, tablets, capsules, caplets, and the like. The active material is prepared by an extraction process by human intervention and is formulated into a composition, such as with other food grade excipients and materials to obtain the desired form.

### BRIEF DESCRIPTION OF THE DRAWINGS

FIG. 1A depicts effect of beta-cryptoxanthin on body weight in high fat diet induced obese rats (n=7).

FIG. 1B depicts effect of beta-cryptoxanthin on visceral fat in high fat diet induced obese rats (n=7).

FIG. 1C depicts effect of beta-cryptoxanthin on liver weight in high fat diet induced obese rats (n=7).

FIG. 2A depicts effect of beta-cryptoxanthin on biochemical parameters (Glucose) in high fat diet induced obese rats (n=7).

FIG. 2B depicts effect of beta-cryptoxanthin on biochemical parameters (Insulin) in high fat diet induced obese rats (n=7).

FIG. 2C depicts effect of beta-cryptoxanthin on biochemical parameters (Leptin) in high fat diet induced obese rats (n=7).

FIG. 2D depicts effect of beta-cryptoxanthin on biochemical parameters (Adiponectin) in high fat diet induced obese rats (n=7).

FIG. 2E depicts effect of beta-cryptoxanthin on biochemical parameters (Tryglyceride (TG)) in high fat diet induced obese rats (n=7).

FIG. 2F depicts effect of beta-cryptoxanthin on biochemical parameters (Total Cholesterol (TC)) in high fat diet induced obese rats (n=7).

FIG. 2G depicts effect of beta-cryptoxanthin on biochemical parameters (LDL-C) in high fat diet induced obese rats (n=7).

FIG. 2H depicts effect of beta-cryptoxanthin on biochemical parameters (HDL-C) in high fat diet induced obese rats (n=7).

FIG. 2I depicts effect of beta-cryptoxanthin on biochemical parameters (Free Fatty Acids (FFA)) in high fat diet induced obese rats (n=7).

FIG. 3A depicts effect of beta-cryptoxanthin on antioxidant status (Serum Total Antioxidant Capacity (TAC)) in high fat diet induced obese rats (n=7).

FIG. 3B depicts effect of beta-cryptoxanthin on antioxidant status (Serum malondialdehyde (MDA)) in high fat diet induced obese rats (n=7).

FIG. 3C depicts effect of beta-cryptoxanthin on antioxidant status (Liver MDA) in high fat diet induced obese rats (n=7).

FIG. 3D depicts effect of beta-cryptoxanthin on antioxidant status (Liver superoxide dismutase (SOD)) in high fat diet induced obese rats (n=7).

FIG. 3E depicts effect of beta-cryptoxanthin on antioxidant status (Liver catalase (CAT)) in high fat diet induced obese rats (n=7).

FIG. 3F depicts effect of beta-cryptoxanthin on antioxidant status (Liver glutathione peroxidase (GSH-Px)) in high fat diet induced obese rats (n=7).

FIG. 4 depicts effect of beta-cryptoxanthin for regulating CCAAT/enhancer-binding protein alpha (C/EBPa), FAS and stearoyl-CoA desaturase (SCD-1).

FIG. 5 depicts effect of beta-cryptoxanthin on retina tissue vascular endothelial growth factor (VEGF), nuclear factor erythroid derived 2-related factor 2 (Nrf-2), nuclear factor kappa light chain enhancer of activated B cells (NFkB), Inducible nitric oxide synthase (iNOS), Intercellular Adhesion Molecule 1 (ICAM-1); and heme-oxygenase 1 (HO-1).

FIG. 6 depicts effect of beta-cryptoxanthin on HO-1 marker in retina tissue.

FIG. 7 depicts effect of beta-cryptoxanthin on ICAM-1 marker in retina tissue.

FIG. 8 depicts effect of beta-cryptoxanthin on iNOS marker in retina tissue.

FIG. 9 depicts effect of beta-cryptoxanthin on nuclear factor kappa-light-chain-enhancer of activated B cells (NFkB) marker in retina tissue.

FIG. 10 depicts effect of beta-cryptoxanthin on Nuclear factor (erythroid-derived 2) (Nrf-2) marker in retina tissue.

FIG. 11 depicts effect of beta-cryptoxanthin on (VEGF) marker in retina tissue.

FIG. 12 depicts effect of beta-cryptoxanthin on liver tissue beta-carotene oxygenase 2 (BCO2), tumor necrosis factor alpha (TNF-α), peroxisome proliferator-activated receptor gamma (PPAR-γ), Nrf-2, NFkB, insulin receptor substrate 1 (IRS-1), HO-1.

FIG. 13 depicts effect of beta-cryptoxanthin on BCO2 marker in liver tissue.

FIG. 14 depicts effect of beta-cryptoxanthin on TNF-α marker in liver tissue.

FIG. 15 depicts effect of beta-cryptoxanthin on PPAR-γ marker in liver tissue

FIG. 16 depicts effect of beta-cryptoxanthin on Nrf-2 marker in liver tissue.

FIG. 17 depicts effect of beta-cryptoxanthin on NFkB marker in liver tissue.

FIG. 18 depicts effect of beta-cryptoxanthin on HO-1 marker in liver tissue.

FIG. 19 depicts effect of beta-cryptoxanthin on IRS-1 marker in liver tissue.

### DETAILED DESCRIPTION

The methods described herein are comprised of identifying a subject in need thereof, administering beta-cryptoxanthin composition(s) in an effective amount(s), and evaluating the effect for treatment, prevention and/or management of cardiometabolic syndrome and their associated risk factor(s). The methods described herein can improve conditions associated with cardiometabolic syndrome such as body weight, lipid profile, insulin resistance, blood glucose, reduction in oxidative stress, and inflammatory markers, to protect vital organs like the eye and liver, when administered to a subject, such as for example who is habituated for high fat diet.

Beta-cryptoxanthin for the compositions as described herein may be obtained by natural resources and are safe for administration and thus useful for nutraceutical purposes.

Cardiometabolic syndrome, also known as syndrome X, increases the risk of developing cardiovascular disease, particularly atherosclerosis, heart failure, dyslipidemia, diabetes, and associated risk factors, which may be caused mainly due to imbalance of calorie intake and energy utilization. One of the most important causes for this is a high fat diet. The syndrome also affects vital body organs such as liver and eye. Therefore it is important to identify methods for treating and preventing it and associated risk factors thereof by administering compositions which are safe for administration and evaluating the effect in subjects in need thereof.

The terminology ‘subject’ is commonly used in the specification to refer to an individual or mammal under test, being treated with compositions herein.

The terminology “subject in need thereof” can include specific individuals or mammals who are habituated to a diet rich in high fat and refined carbohydrates, thus lacking in fibers. Such subjects are at high risk of developing cardiometabolic syndrome or symptoms for associated risk factors and/or may be suffering from cardiometabolic syndrome, because of developing abdominal obesity.

Abdominal obesity drives the progression of multiple risk factors directly, through secretion of excess free fatty acids and inflammatory adipokines, and decreased secretion of adiponectin (Després J P et al, 1990; Pouliot M C, 1992; Kissebah A H et al, 1989; Carey V J, 1997; Turkoglu C et al, 2003). Significant effects of abdominal obesity can be dyslipidaemia and insulin resistance, which can provide an indirect, though clinically important, link to the genesis and progression of atherosclerosis and cardiometabolic risk. Excess abdominal obesity is accompanied by elevated levels of C-reactive protein (CRP) and free fatty acids (FFAs), as well as decreased levels of adiponectin. Elevated CRP is an indicator of inflammation. Abdominal obesity may be associated with the inflammation cascade, with adipose tissue expressing a number of inflammatory cytokines. Inflammation is now believed to play a role in the development of atherosclerosis and type 2 diabetes. Elevated levels of CRP are considered to be predictive of cardiovascular disease and insulin resistance.

Elevated FFA levels appear to play a significant role in the cause of insulin resistance. It has been suggested that elevated FFAs and intracellular lipids inhibit the insulin signaling mechanism, leading to decreased glucose transport to muscle. Adiponectin is an adipose tissue-specific circulating protein which is involved in the regulation of lipid and glucose metabolism. Adiponectin has been shown to be reduced in adults with obesity and type 2 diabetes. Such components help to explain why excess abdominal obesity is considered to be a significant risk to cardiovascular and metabolic health.

Inflammation is part of the complex biological response of vascular tissues to harmful stimuli, such as pathogens, damaged cells, or irritants. Chronic inflammation is widely observed in obesity. Understanding the molecular basis of inflammation has led to the identification of markers that may also serve as new targets of therapy in the management of associated cardiometabolic syndrome disease in obese person. The obese commonly have many elevated markers of inflammation, including: Interlukins (IL 6, 8 and 18), TNF-α (Tumor necrosis factor-alpha), CRP (C-reactive protein), Insulin, Blood glucose, and Leptin. Inflammatory markers have been shown to predict future cardiovascular events in subjects with and without established cardiovascular disease (CVD).

Low-grade chronic inflammation is characterized by a two- to threefold increase in the systemic concentrations of cytokines such as TNF-α, IL-6, and/or CRP. TNF's primary role is to regulate the immune cells and induce inflammation. TNFα-induced reductions in insulin sensitivity in adipocytes are partly responsible for the increased free fatty acid production and hypertriglyceridaemia characteristic of abdominal obesity. Leptin responds specifically to adipose-derived inflammatory cytokines. Hyperglycemia induces IL-6 production from endothelial cells and macrophages. Meals high in saturated fat, as well as meals high in calories have been associated with increases in inflammatory markers.

Liver plays an important role in metabolism activities and it is an important site of fat metabolism. When this function is impaired due to a variety of reasons, fat accumulation occurs in the liver, which may result in cirrhosis and/or increased risk of other cardiometabolic syndromes such as for example diabetes, hypertension, disturbed lipid profile, and/or one or more risk factors associated with these syndromes, or in combination with other associated conditions.

As per one embodiment, the methods described herein are comprised of administering beta-cryptoxanthin compositions to a subject in need thereof, in an effective amount, and evaluating its effect on risk factors associated with cardiometabolic syndrome. Beta-cryptoxanthin compositions herein may be administered by oral route, in combination with antioxidant or other nutrients, using oil vehicle for suspending the composition. The oil used in the composition is selected from the group consisting of rape seed oil, corn oil, sunflower oil and like thereof.

According to one embodiment, methods and compositions as described herein are directed to treating macular degeneration in a subject in need thereof comprising essentially of administering therapeutically active amounts of beta-cryptoxanthin either alone or in combination with antioxidant or an oil.

As per one embodiment, the compositions and methods herein can improve (e.g. reduce) risk factors associated with cardiometabolic syndrome, such as body weight, lipid profile, body glucose, and/or insulin resistance, when administered to a subject, such as for example a subject who is fed with a high fat diet.

In another embodiment, a method for treating dyslipidema, comprising identifying a subject with elevated triglycerides levels, elevated serum LDL levels, or reduced HDL levels and accordingly administering a therapeutically effective amount of a composition consisting essentially of beta-cryptoxanthin either alone or in combination of pharmaceutically acceptable excipients.

In further embodiment, methods described herein are comprised of administering effective amount of a composition to a subject in need thereof for improving insulin sensitivity. The composition may be beta-cryptoxanthin either alone or in combination of pharmaceutically acceptable excipients.

The terminology ‘high fat diet’ as used in the specification includes a diet with food typically containing about 32 to 60% of calories from fat. Such diets with 60 kcal % fat are commonly used to induce obesity in rodents since animals tend to gain weight more quickly, thereby allowing researchers to screen their compounds after a shorter period of time.

The type of fat is also considered when choosing a high-fat diet for an animal study. Many high-fat diets used in laboratory animal research contain more saturated fat such as lard, beef tallow, or coconut oil and these diets are quite capable of inducing obesity in susceptible strains.

As per one embodiment, methods described herein are comprised of administering an effective amount of beta-cryptoxanthin compositions to treat hyperlipidemia in a subject in need thereof by lowering total cholesterol, low density lipoproteins and/or triglycerides.

According to one embodiment, methods and compositions described herein are directed to lowering free fatty acid levels, and/or visceral fat, along with liver weight and body weight, when administered to a subject, who may be fed with a high fat diet.

According to one embodiment, methods and beta-cryptoxanthin compositions herein are also used to treat and/or evaluate their effect on expression of inflammatory markers and/or oxidative stress markers. It is observed that beta-cryptoxanthin compositions herein and methods of use thereof reduce inflammatory markers.

According to one embodiment, beta-cryptoxanthin compositions and methods of use thereof can protect organs, which may be at risk because of cardiometabolic syndrome, such as the eye and liver by reducing oxidative stress and/or inflammatory manifestations.

In one embodiment, beta-cryptoxanthin compositions and methods herein are directed to treat and/or be evaluate for their effect on the management of risk factors associated with cardiometabolic syndrome, in a subject, in need thereof, when administered in an effective amount(s).

In another embodiment, as per methods described herein, beta-cryptoxanthin compositions described herein are evaluated for effectiveness in significantly overcoming the cardiometabolic syndrome and associated risk factors such as body weight, body fats, lipid profile, blood glucose and the like.

The methods as described herein are comprised of evaluating effect of beta-cryptoxanthin composition on prevention and treatment of cardiometabolic syndrome through gene expression study on adipocyte cell system. CCAAT/enhancer binding proteins (C/EBP) are involved in different cellular responses, such as in the control of cellular proliferation, growth and differentiation, in metabolism, and in immunity. Their expression is regulated at multiple levels, including hormones, mitogens, cytokines, nutrients, and other factors. The encoded protein has been shown to bind to the promoter and modulate the expression of the gene encoding leptin, a protein that plays an important role in body weight homeostasis. C/EBPa is involved in adipogenesis and with normal adipocyte function. C/EBPα promotes adipogenesis by inducing the expression of PPARγ.

Fatty Acid Synthase main function is to catalyze the synthesis of palmitate from acetyl-CoA and malonyl-CoA, in the presence of nicotinamide adenine dinucleotide (NADPH), into long-chain saturated fatty acids. The role of fatty acid synthase is implicated in the regulation of fatty acid synthesis and net accumulation of lipid in liver and adipose tissue. The role of fatty acid synthase is implicated in the regulation of fatty acid synthesis and net accumulation of lipid in liver and adipose tissue. FAS expression was controlled possibly at transcriptional level through peroxisome proliferator-activated receptor (PPARs) and sterol regulatory element-binding proteins (SREBPs) mediated signaling path way.

Stearoyl-CoA desaturase-1 (SDC-1) is a key enzyme in fatty acid metabolism. The elevated expression levels of SCD1 are found to be correlated with obesity. This phenomenon depends on increased expression of fatty acid biosynthetic enzymes that produce required fatty acids in large quantities. Alteration in SCD1 expression changes the fatty acid profile of these lipids and produces diverse effects on cellular function. High SCD1 expression is correlated with metabolic diseases such as obesity and insulin resistance, whereas low levels are protective against these metabolic disturbances. However, SCD1 is also involved in the regulation of inflammation and stress in distinct cell types, including β-cells, adipocytes, macrophages, endothelial cells, and myocytes.

Particularly the effect of the beta-cryptoxanthin compositions are evaluated for fat accumulation, modulation of collagen and transforming growth factor beta (TGF-beta) signaling pathways in high fat fed diet (HFFD) rats. The effect of beta-cryptoxanthin compositions are also evaluated on vascular endothelial growth factor (VEGF), nuclear factor erythroid 2 (NrF2), nuclear factor kappa-light-chain-enhancer of activated B cells (NFkB), Inducible nitric oxide synthase (INOS), intercellular adhesion molecule 1 (ICAM-1) and heme oxygenase 1 (HO-1) pathways in retinal tissue of HFD treated rats.

Additionally the beta-cryptoxanthin compositions are evaluated on beta-carotene oxygenase 2 (BCO2), tumor necrosis factor alpha (TNF-α), peroxisome proliferator-activated receptor gamma (PPAR-γ), nuclear factor erythroid 2 (Nrf-2), nuclear factor kappa-light-chain-enhancer of activated B cells (NFkB), insulin receptor substrate 1 (IRS-1), heme oxygenase 1 (HO-1) pathways in liver tissue of HFD treated rats.

The compositions herein include beta-cryptoxanthin concentrates of high purity. In particular, beta-cryptoxanthin concentrates containing about 10-80% by weight total xanthophylls (total carotenoids) of which the trans-beta-cryptoxanthin content is about 75-98% by weight and the remaining including zeaxanthin, trans-capsanthin, beta-carotene and trace amounts of other carotenoids. The concentrates are particularly useful as dietary supplements for nutrition and health promoting benefits.

Processes are described for the preparation of the beta-cryptoxanthin concentrate from plant oleoresin, especially from Capsicum oleoresin. The process includes the steps of admixing the oleoresin with alcohol solvents, saponifying the xanthophyll esters, washing and purifying by eluting the crude xanthophyll viscous concentrate on a silica gel column, and purifying further by washings to obtain high purity trans-beta-cryptoxanthin enriched concentrate crystals.

In some embodiments, a process is described for the isolation of beta-cryptoxanthin crystals containing at least or about 80% by weight of total xanthophylls (total carotenoids) in free form, out of which the trans-beta-cryptoxanthin content is at or about or at least 98.5% by weight, the remaining including trace amounts of zeaxanthin, trans-capsanthin, beta-carotene and other carotenoids derived from oleoresin and extracts of plant materials such as Capsicum sources.

In some embodiments, a process is described for the preparation of beta-cryptoxanthin crystals containing at or about or at least 40% by weight of total carotenoids, out which the trans-beta-cryptoxanthin is at or about or at least 90% by weight, the remaining including trace amounts of zeaxanthin, trans-capsanthin, beta-carotene and other carotenoids derived from oleoresin and extracts of plant materials such as Capsicum sources.

In some embodiments, a process is described for the preparation of beta-cryptoxanthin crystals containing at or about or at least 10% by weight of total carotenoids, out of which the trans-beta-cryptoxanthin is at or about or at least 75% by weight, the remaining including zeaxanthin, trans-capsanthin, beta-carotene and traces amounts of other carotenoids derived from oleoresin and extracts of plant materials such as Capsicum sources.

In some embodiments, a process is described for the preparation of beta-cryptoxanthin crystals containing total carotenoids at or about 10 to at or about 80% by weight, out of which the trans-beta-cryptoxanthin content is in the range of at or about 75 to at or about 98% by weight, the rest including zeaxanthin, trans-capsanthin, beta-carotene and trace amounts of other carotenoids derived from a starting material like saponified Capsicum extract.

In some embodiments, a process is described for the preparation of high purity beta-cryptoxanthin from capsicum oleoresin or saponified capsicum extract. In some embodiments, residual solvent-free beta-cryptoxanthin crystals, in which trans-beta-cryptoxanthin form the major ingredient in the total carotenoids. In one embodiment, processes herein provide recovery of carotene hydrocarbon fractions rich in beta-carotene.

In some embodiments, the process for obtaining high purity trans-beta-cryptoxanthin includes:
saponification of esterified xanthophylls in Capsicum extract, which results in free xanthophylls and which is purified by washing with acidified water, followed by drying to obtain a carotenoid mass;
treating the carotenoid mass with non-polar solvent under stirring, followed by filtration and concentration to obtain a mass;
subjecting the mass to column chromatography using silica gel and elution using non-polar solvent to remove beta-carotene;
eluting the column with non-polar solvent containing at or about 2% polar solvent, and obtaining an eluent after concentration of a concentrate showing at or about 10% total carotenoids by weight, of which trans-beta-cryptoxanthin comprises at or about 75% by weight;
treating the above concentrate with ethanol under stirring, followed by cooling to at or about 10° C. and filtering to obtain a semi-purified crystalline mass showing total xanthophylls at or about 40% by weight, of which trans-beta-cryptoxanthin comprises at or about 98%>by weight; and
washing the crystalline mass with hexane containing about 20% ethyl acetate, cooling to at or about −10° C. and filtering to obtain a high purity crystalline material showing at or about 80% total xanthophylls by weight, of which trans-beta-cryptoxanthin comprises at or about 98.5%>by weight.

In some embodiments, a process is described for the preparation of a beta-cryptoxanthin enriched concentrate from plant material comprising at or about 10-80% by weight total xanthophylls, of which at or about 75-98% by weight is trans-beta-cryptoxanthin. The process comprises: (a) mixing an oleoresin of plant material comprising xanthophylls esters with an aliphatic alcoholic solvent; (b) saponifying the xanthophylls esters present in the oleoresin with an alkali at an elevated temperature; (c) removing the aliphatic alcoholic solvent followed by addition of water to obtain a diluted resultant mixture; (d) adding a diluted organic acid to the diluted resultant mixture to form a water layer and a precipitated xanthophylls mass; (e) removing the water layer and washing the precipitated xanthophylls mass with a polar solvent; (f) drying the precipitated xanthophylls mass to obtain a crude xanthophylls mass; (g) washing the crude xanthophylls mass with a non-polar solvent and concentrating the non-polar solvent washings to obtain a concentrated crude xanthophylls mass; (h) transferring the concentrated crude xanthophylls mass to a silica gel column and washing with a non-polar solvent; (i) eluting the column with a mixture of non-polar and polar solvent and concentrating the elutions to obtain a trans-beta-cryptoxanthin-rich xanthophylls concentrate; (j) admixing the trans-beta-cryptoxanthin-rich xanthophylls concentrate with an aliphatic alcohol and then cooling; and (k) filtering and drying the trans-beta-cryptoxanthin-rich xanthophylls concentrate to obtain a purified trans-beta-cryptoxanthin concentrate.

In some embodiments, the xanthophylls esters in the oleoresin of plant material in step (a) are present at or about 6-8% by weight. In some embodiments, the aliphatic alcohol of step (a) or (j) is selected from the group consisting of ethanol, methanol, isopropyl alcohol, and mixtures thereof.

In some embodiments, the ratio of oleoresin to alcohol in step (a) ranges from at or about 1:0.25 to at or about 1:1 weight/volume. In some embodiments, the alkali of step (b) is selected from the group consisting of sodium hydroxide, potassium hydroxide, and mixtures thereof. In some embodiments, the ratio of oleoresin to alkali in step (b) ranges from at or about 1:0.25 to at or about 1:0.5 weight/weight.

In some embodiments, the elevated temperature of step (b) ranges from at or about 75 to at or about 85° C. In some embodiments, the addition of water in step (c) is at or about 5 times that of the oleoresin (weight/weight).

In some embodiments, the diluted organic acid of step (d) is acetic acid or phosphoric acid. In some embodiments, the diluted organic acid of step (d) is a solution of at or about 20% to at or about 50% organic acid.

In some embodiments, the polar solvent of step (e) is water.

In some embodiments, the non-polar solvent of steps (g), (h), and (i) is selected from the group consisting of a hexane, a pentane, a heptane, and mixtures thereof.

In some embodiments, the crude xanthophylls mass and non-polar solvent of step (g) is in a ratio of at or about 1:10 to at or about 1:15 weight/volume. In some embodiments, the concentrated crude xanthophylls mass of step (g) comprises beta-carotene, trans-beta-cryptoxanthin, trans-capsanthin, zeaxanthin, and trace amounts of other carotenoids, such as capsorubin or violaxanthin.

In some embodiments, the concentrated crude xanthophylls mass and the non-polar solvent of step (h) are in a ratio of at or about 1:5 to at or about 1:8 weight/volume. In some embodiments, a carotene concentrate is obtained by distilling the non-polar solvent washing of step (h). In some embodiments, the carotene concentrate is beta-carotene.

In some embodiments, the polar solvent of step (i) is selected from the group consisting of a propanone, a pentanone, and mixtures thereof. In some embodiments, the non-polar solvent and polar solvent of step (i) are in a ratio of at or about 95:5 to at or about 98:2. In some embodiments, the trans-beta-cryptoxanthin-rich xanthophylls concentrate of step (i) comprises at or about or at least 10% by weight of total xanthophylls, of which trans-beta-cryptoxanthin content is at or about or at least 75% by weight.

In some embodiments, the cooling in step j) is performed at or about 10° C. In some embodiments, the purified trans-beta-cryptoxanthin concentrate of step (k) comprises at or about or at least 40% by weight of total xanthophylls, of which trans-beta-cryptoxanthin content is at or about or at least 90% by weight.

In some embodiments, the process further comprises a step (1): washing the purified trans-beta-cryptoxanthin concentrate with a mixture of non-polar and ester solvent and cooling for precipitation to obtain high purity trans-beta-cryptoxanthin crystals. In some embodiments, the high purity trans-beta-cryptoxanthin crystals of step (1) comprises at or about or at least 80% by weight of total xanthophylls, of which trans-beta-cryptoxanthin content is at or about or at least 98% by weight. In some embodiments, the ester solvent of step (1) is ethyl acetate and the non-polar solvent of step (1) is hexane. In some embodiments, the non-polar solvent and ester solvent of step (1) are in a ratio of at or about 80:20 to at or about 90:10. In some embodiments, the temperature for cooling in step (1) is at or about −10° C.

In some embodiments, a process is described for the preparation of a beta-cryptoxanthin enriched concentrate from plant material comprising at or about or at least 80% by weight total xanthophylls, of which at or about or at least 98% by weight is trans-beta-cryptoxanthin, the process comprising: (a) mixing an oleoresin of plant material comprising xanthophylls esters with ethanol, wherein the ratio of oleoresin to ethanol is at or about 1:1 weight/volume; (b) saponifying the xanthophylls esters present in the oleoresin with potassium hydroxide without addition of water, wherein the ratio of oleoresin to potassium hydroxide is at or about 1:0.25 weight/weight; (c) applying heat to the oleoresin to elevate the temperature up to reflux at or about 80-85° C.; (d) agitating the oleoresin for about 3 to 5 hours at or about 80-85° C.; (e) evaporating the ethanol under vacuum followed by addition of water at or about 5 times that of the oleoresin (weight/weight) to obtain a diluted resultant mixture and agitating for at or about 1 hour; (f) neutralizing the diluted resultant mixture with about 25% acetic acid to form a water layer and a precipitated xanthophylls mass; (g) separating the water layer from the precipitated xanthophylls mass and washing the mass with water to remove soaps and other polar soluble materials; (h) drying the precipitated xanthophylls mass under vacuum to obtain a crude xanthophylls mass; (i) washing the crude xanthophylls mass with at or about 1:10 hexane (weight/volume) and concentrating the hexane washings to obtain a concentrated crude xanthophylls mass; j) transferring the concentrated crude xanthophylls mass to a silica gel column at a ratio of at or about 1:5 (weight/weight) and eluting with hexane to obtain a carotene fraction; (k) washing the column with at or about 98:2 hexane to acetone and concentrating the washings to obtain a trans-beta-cryptoxanthin-rich xanthophylls concentrate; (l) admixing the trans-beta-cryptoxanthin-rich xanthophylls concentrate with at or about 1:2 ethanol under stirring and then cooling at or about 10° C. for about 8 hours; (m) filtering and drying the trans-beta-cryptoxanthin-rich xanthophylls concentrate under vacuum to obtain a purified trans-beta-cryptoxanthin concentrate; and (n) washing the purified trans-beta-cryptoxanthin concentrate with at or about 80:20 hexane:ethyl acetate and cooling to at or about −10° C. for about 18 hours for precipitation to obtain high purity trans-beta-cryptoxanthin crystals.

In some embodiments, the total xanthophylls of the processes comprise byproducts selected from zeaxanthin, trans-capsanthin, beta-carotene, trace amounts of other carotenoids, and any combinations thereof.

In some embodiments, the plant material used in the processes or to derive the beta-cyrptoxanthin concentrates is selected from at least one of the group consisting of fruits, vegetables, and mixtures thereof. In some embodiments, the plant material is from a capsicum.

In some embodiments, beta-cryptoxanthin concentrates herein may be administered in a dosage form selected from beadlets, microencapsulated powders, oil suspensions, liquid dispersions, capsules, pellets, ointments, soft gel capsules, tablets, chewable tablets or lotions/liquid preparations. In some embodiments, the beta-cryptoxanthin concentrate is added to or as part of another composition.

In some embodiments, compositions herein includes a beta-cryptoxanthin concentrate derived from plant material, wherein the concentrate comprises at or about or at least 10% by weight total xanthophylls, of which at or about or at least 75% by weight is trans-beta-cryptoxanthin. In some embodiments, the total xanthophylls comprise by-products selected from zeaxanthin, trans-capsanthin, beta-carotene, trace amounts of other carotenoids such as capsorubin or violaxanthin, and combinations thereof. In some embodiments, the composition further comprises a pharmaceutically acceptable ingredient or a food grade ingredient.

In some embodiments, the total xanthophylls of the beta-cryptoxanthin concentrate comprise by-products selected from the group consisting of zeaxanthin, trans-capsanthin, beta-carotene, trace amounts of other carotenoids such as capsorubin or violaxanthin, and combinations thereof.

In some embodiments, a beta-cryptoxanthin concentrate is provided, which contains at or about 10-80% by weight total xanthophylls, of which at or about 75-98% by weight is trans-beta-cryptoxanthin, the remaining including zeaxanthin, trans-capsanthin, beta-carotene and trace amounts of other carotenoids, derived from oleoresin or extract of plant material and which is useful for nutrition and health care. In some embodiments, the concentrate comprises at or about or at least 10% by weight total xanthophylls, of which at or about or at least 75% by weight is trans-beta-cryptoxanthin. In some embodiments, the concentrate comprises at or about or at least 40% by weight total xanthophylls, of which at or about or at least 90% by weight is trans-beta-cryptoxanthin.

In some embodiments, the concentrate comprises at or about or at least 80% by weight total xanthophylls, of which at or about or at least 98% by weight is trans-beta-cryptoxanthin.

The plant material is derived from sources including, but not limited to, fruits and vegetables. In some embodiments, the plant material is derived from capsicum. Capsicum is a genus of flowering plants that includes several varieties of peppers, such as but not limited to red peppers, and the word “capsicum” is also used interchangeably in several parts of the world when referring to peppers. The capsicum oleoresin described herein also includes paprika oleoresin.

In some embodiments, beta-cryptoxanthin enriched concentrates herein can be formulated in a dosage form including, but not limited to, beadlets, microencapsulated powders, oil suspensions, liquid dispersions, capsules, pellets, ointments, soft gel capsules, tablets, chewable tablets or lotions/liquid preparations. The beta-cryptoxanthin enriched concentrates herein can also be provided in a food or feed (including liquid or solid) composition. It will be appreciated that suitable delivery methods include, but are not limited to, oral, parenteral, subcutaneous, intravenous, intramuscular, intraperitoneal, transdermal, intracranial, or buccal administration.

Compositions herein comprising the trans-beta-cryptoxanthin enriched concentrates herein include in some embodiments one or more suitable pharmaceutically acceptable ingredients or food grade ingredients such as, but not limited to, carriers, binders, stabilizers, excipients, diluents, pH buffers, disintegrators, solubilizers and isotonic agents.

Compositions herein may include an “effective amount” of the trans-beta-cryptoxanthin enriched concentrates. An “effective amount” refers to an amount effective, at a dose and in certain circumstances for a period of time to achieve a desired result, for example in methods of treatment or prevention of symptoms for use in such methods. The effective amount may vary according to factors such as the disease state, age, sex, and weight of the individual.

The beta-cryptoxanthin compositions herein includes an active material present including beta-cryptoxanthin (BCX), which is extracted for example from paprika oleoresin by saponification followed by purification through column chromatography. Compositions herein are enriched with trans-beta-cryptoxanthin. In an embodiment, the extract is suspended in a suitable oil medium to obtain 5% oil suspension. In an embodiment, the suspension was evaluated in animal model described herein below. For human consumption, the compositions herein include final formulations into powders, granules, beadlets, and can be administered by oral solid dosage forms such as tablets, capsules.

In an embodiment, an effective amount herein relates to the amount of BCX present in the composition.

In an embodiment, a daily dose duration can range from at or about 3 months to at or about 2 years, or till the desired effect is achieved in a subject. It will be appreciated that there may be no fixed time period for the daily doses as it may be less or longer than such range. It will also be appreciated that the dose may be given continuously daily during this period or the administration can be stopped after obtaining a desired effect in a subject, and can also be restarted again as needed. It is appreciated that dose periods herein include the experiment durations or by general volunteer study period which can be extended to 12 months.

In an embodiment, an effective daily dose includes a range of at or about 250 micrograms to at or about 30 mg/kg body weight.

In an embodiment, an effective daily dose includes a range of at or about 150 micrograms to at or about 20 mg/kg body weight.

In an embodiment, an effective daily dose includes a range of at or about 200 micrograms to at or about 10 mg/kg body weight.

Compositions and methods of preparing beta-cryptoxanthin are disclosed in Applicant's copending published application US 2015/0361040, which is herewith incorporated by reference.

The following examples are given by the way of illustration and therefore should not be construed to limit the scope of the disclosures or innovations herein.

While the compositions and methods have been described in terms of illustrative embodiments, certain modifications and equivalents will be apparent to those skilled in the art and are intended to be included within the scope of the compositions and methods herein. The details and advantages of which are explained hereunder in greater detail in relation to non-limiting exemplary illustrations.

### EXAMPLES

### Process for Extraction of Beta-Cryptoxanthin from Paprika Oleoresin

### Example 1

A weighed quantity of 100 g of Paprika oleoresin containing 7.72% total xanthophylls and a color value of 1,23,515 units (HPLC profile of the oleoresin: beta-15.36% carotene; 10% trans-beta-cryptoxanthin; 7.6% zeaxanthin; and 31.50% trans-capsanthin) was mixed with 100 ml ethanol and 25 g potassium hydroxide pellet. The reaction mixture was heated to a temperature of 80-85° C. with stirring. This saponification process was maintained for 3-5 hours at 80-85° C. with gentle agitation. The reaction mixture was cooled, and then ethanol was distilled out from the mass. A measured volume of water (700 ml) was added to the reaction mixture and agitated for 1 hour. The solution was neutralized with 25% acetic acid solution. The water layer from the mass was separated, and the mass was washed thrice with water. The mass was collected and dried under vacuum. The saponifed mass concentrate obtained was 124 g with a total xanthophylls content of 3.73% by weight (HPLC profile of the saponifed mass concentrate: 22.53% beta-carotene; 12.32% trans-beta-cryptoxanthin; 11% zeaxanthin; and 29.3% trans-capsanthin).

The saponified mass concentrate was washed two times with 1:10 hexane (wt/vol) at room temperature under stirring, filtered, and the combined filtrate concentrated to obtain a concentrated crude xanthophylls mass. The concentrated crude xanthophylls mass (hexane concentrate) obtained was 72 g with a total xanthophylls content of 3.2% (HPLC profile of the concentrated crude xanthophylls mass: 39.01% beta-carotene; 21.78% trans-beta-cryptoxanthin; 5.70% zeaxanthin; and 9.86% trans-capsanthin).

The residue (saponified xanthophylls) remaining after hexane wash was 22 g, which on analysis showed a total xanthophylls content of 10% (HPLC profile of the residue: 0.7% beta-carotene; 3.43% trans-beta-cryptoxanthin; 15.32% zeaxanthin; and 52.84% trans-capsanthin).

The hexane concentrate was dissolved in a minimum amount of hexane and subjected to column chromatographic separation. The column was packed with 1:5 concentrate to Silica 100-200 mesh (wt/wt). The column was washed with hexane, and the separated band was collected and concentrated (yield 55 g with a total xanthophylls content of 2.3%, HPLC profile: 99.8% beta-carotene). The column was then eluted with 98:2 hexane:acetone (v/v), and the eluent collected and concentrated. This concentrate layer was enriched with beta-cryptoxanthin (yield 5.2 g with a total xanthophylls content of 10.26%, HPLC profile: 75.56% trans-beta-cryptoxanthin). Finally, the column was washed with acetone and the washings concentrated to obtain trans-capsanthin enriched residue.

### Example 2

A quantity of approximately 100 g of Paprika oleoresin containing 6.50% total xanthophylls and a color value of 1,05,457 units (HPLC profile of the oleoresin: 15.73% beta-carotene; 9.07% trans-beta-cryptoxanthin; 10.54% zeaxanthin and 31.38% trans-capsanthin) was mixed with 100 ml ethanol and 25 g potassium hydroxide pellet. The reaction mixture was heated to a temperature of 80-85° C. with stirring. This saponification process was maintained for 3-5 hours at 80-85° C. with gentle agitation. The reaction mixture was cooled, and then ethanol was distilled out from the mass. A measured volume of water (700 ml) was added to the reaction mixture and agitated for 1 hour. The solution was neutralized with 40% acetic acid solution. The water layer from the mass was separated, and the mass was washed thrice with water. The mass was collected and dried under vacuum. The saponified mass concentrate obtained was 126 g with a total xanthophylls content of 3.73% by weight (HPLC profile of the saponified mass concentrate: 16.34% beta-carotene; 9.41% trans-beta-cryptoxanthin; 8.57% zeaxanthin; and 24.35% trans-capsanthin).

The saponified mass concentrate was washed two times with 1:10 hexane (wt/vol) at room temperature under stirring, filtered, and the combined filtrate concentrated to obtain a concentrated crude xanthophylls mass. The concentrated crude xanthophylls mass (hexane concentrate) obtained was 76.15 g with a total xanthophylls content of 3.26% (HPLC profile of the concentrated crude xanthophylls mass: 31.80% beta-carotene; 14.04% trans-beta-cryptoxanthin; 4.35% zeaxanthin; and 8.70% trans-capsanthin).

The residue (saponified xanthophylls) remaining after hexane wash was 16 g, which on analysis showed a total xanthophylls content of 11% (HPLC analysis of the residue: 1.22% beta-carotene; 0.75% trans-beta-cryptoxanthin; 33.29% zeaxanthin; and 29.99% trans-capsanthin).

The hexane concentrate was dissolved in a minimum amount of hexane and subjected to column chromatographic separation. The column was packed with 1:5 concentrate to Silica 100-200 mesh (wt/wt), eluted with hexane, and the first band separated was collected and concentrated (yield 54.72 g with a total xanthophylls content of 1.08%, HPLC profile: 85.88% beta-carotene). The column was then eluted with 98:2 hexane:acetone (v/v) collecting the eluent fraction and concentrated. This fraction was enriched with beta-cryptoxanthin, yielding 4.02 g with a total xanthophylls content of 9% (HPLC profile of the enriched beta-cryptoxanthin concentrate: 76.04% trans-beta-cryptoxanthin). Finally the column was washed with acetone.

The 4.02 g fraction concentrate was stirred with 1:2 ethanol (wt/vol) for 1 hr, chilled for 8 hrs at 10° C., filtered, and the precipitate dried under vacuum. The yield obtained was 0.42 g crystalline precipitate with a total xanthophylls content of 42.45%. The HPLC profile of the crystalline precipitate showed 98.3% trans-beta-cryptoxanthin.

### Example 3

A weighed quantity of Paprika oleoresin (100 g) containing 6-8%>by weight total xanthophylls and a color value of 100,000 units (HPLC profile of the oleoresin: 15.36% beta-carotene; 10% trans-beta-cryptoxanthin; 7.6% zeaxanthin; and 31.50% trans-capsanthin) was mixed with 100 ml ethanol and 25 g potassium hydroxide pellet. The reaction mixture was heated to a temperature of 80-85° C. with stirring. This saponification process was maintained for 3-5 hours at 80-85° C. with gentle agitation. The reaction mixture was cooled and then ethanol was distilled off from the mass under vacuum. A measured volume of water (700 ml) was added to the reaction mixture and agitated for 1 hour. The solution was neutralized with 25% acetic acid solution. The water layer from the mass was removed, and the mass was washed thrice with water. The mass was collected and dried under vacuum. The saponified mass concentrate obtained was 121.75 g with a total xanthophylls content of 4.92% by wt (HPLC profile of the saponified mass concentrate: 21.76% beta-carotene; 12.74% trans-beta-cryptoxanthin; 10.13% zeaxanthin; and 38.25% trans-capsanthin).

The saponified mass concentrate was washed two times with 1:10 hexane (wt/vol) at room temperature under stirring, filtered, and the combined filtrate concentrated to get a concentrated crude xanthophylls mass. The concentrated crude xanthophylls mass (hexane concentrate) obtained was 85.81 g with a total xanthophylls content of 3.21% by wt (HPLC profile of the concentrated crude xanthophylls mass: 35.28% beta-carotene; 19.65% trans-beta-cryptoxanthin; 3.99% zeaxanthin; and 13.88% trans-capsanthin).

The residue (saponified xanthophylls) remaining after hexane wash was 25.65 g, which on analysis showed a total xanthophylls content of 10.42% by wt (HPLC analysis of the residue: 0.7% beta-carotene; 1.24% trans-beta-cryptoxanthin; 18.98% zeaxanthin; and 52.32% trans-capsanthin.

The hexane concentrate was dissolved in minimum amount of hexane and subjected to column chromatographic separation. The column was packed with 1:5 concentrate to Silica gel 100-200 mesh (wt/wt), eluted with 5-8 volumes of hexane, and the first band separated was eluted and concentrated (yield 55 g with a total xanthophylls content of 2.29% wt, HPLC profile: 99% beta-carotene). The column was then eluted with 98:2 hexane:acetone (vol/vol) collecting the eluent fraction and concentrated. This concentrate was enriched with beta-cryptoxanthin, yielding 9.06 g with a total xanthophylls content of 6.12% by wt (HPLC profile of the enriched beta-cryptoxanthin concentrate: 71.80% trans-beta-cryptoxanthin). Finally the column was eluted with acetone.

The 9.06 g beta-cryptoxanthin concentrate was stirred with 1:2 ethanol (wt/vol) for 1 hr, chilled for 8 hours at 10° C., filtered, and the precipitate dried under vacuum. The yield obtained was 0.5 g with a total xanthophylls content of 42.35% by wt. The HPLC profile of the crystal showed 98.3% trans-beta-cryptoxanthin content.

The 0.5 g beta-cryptoxanthin precipitate was dissolved in a minimum amount of 80:20 hexane:ethyl acetate (vol/vol) and chilled for 18 hrs at −10° C., filtered, and the precipitate dried under vacuum. The yield obtained was 0.03 g with a total xanthophylls content of 80% and HPLC profile for trans-beta-cryptoxanthin of 98.50%.

### Example 4

### In-Vitro Evaluation of Beta-Cryptoxanthin Composition

3T3L1 murine adipocytes model system was used to understand basic cellular mechanisms associated with diabetes, obesity and related disorders. Nutrigenomics study was carried out to evaluate effect of beta-cryptoxanthin on adipocyte cells, particularly differentiated 3T3-L1 cells, the study was based on real-time polymerase chain reaction (Real time PCR) with dose 12.5 ug/mL. The effect of beta-cryptoxanthin on Adipocyte differentiation and its activity for bio markers PPARG, SCD-1, acetyl Coa carboxylase (ACC), SREBP-1, C/EBPa and FAS were evaluated.

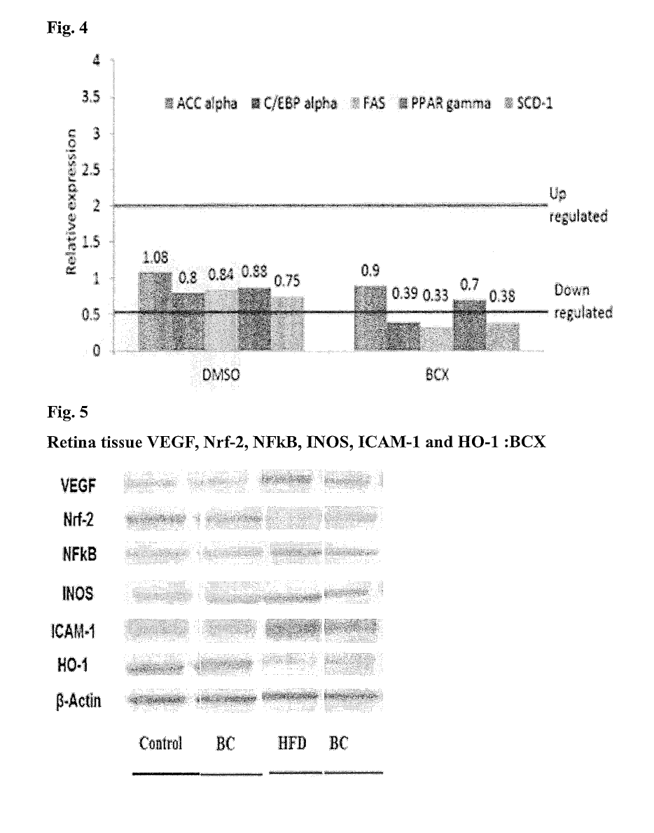

It was observed that BCX down regulated C/EBP and fatty acid synthase (FAS). BCX also down regulated Stearoyl-CoA desaturase-1 (SCD-1), wherein down-regulation of SCD-1 is an important component of leptin's metabolic actions. See FIG. 4, where each set of bar graphs for tests with BCX and without BCX (DMSO) correspond from the leftside bar respectively with the heading legend of the factors tested (i.e. for the bar results of BCX, 0.9 for ACC alpha, 0.39 for C/EPB alpha, 0.33 for FAS, 0.7 for PPAR gamma, and 0.38 for SCD-1).

### Example 5

### In-Vivo Evaluation of Beta-Cryptoxanthin Composition in Rat

a. Effect of Beta-Cryptoxanthin Compositions on Cardiometabolic Markers, Fat Accumulation, Modulation of Collagen and TGF-Beta Signaling Pathways in High Fat Fed Diet (HFFD) Rats

### Animals

Male Sprague Dawley rats (7 rats/group, age: 8 week, weight: 180±20 g) were housed in a controlled environment with a 12:12-h light-dark cycle at 22° C. and provided with rat chow and water ad libitum. All experiments were conducted under the National Institutes of Health's Guidelines for the Care and Use of Laboratory Animals and approved by the Ethics Committee of the Veterinary Control Institute. After acclimation for 2 weeks, the rats were divided into four groups: Rats were randomly divided into the following groups: (1) Control, (2) High Fat Diet (HFD) (42% of calories from fat), (3) control+beta-cryptoxanthin (2.5 mg/kg) 4) HFD (42% of calories from fat)+beta-cryptoxanthin (2.5 mg/kg) was administered daily as supplement for 12 weeks.

### i) Effect of BCX on Visceral Fat:

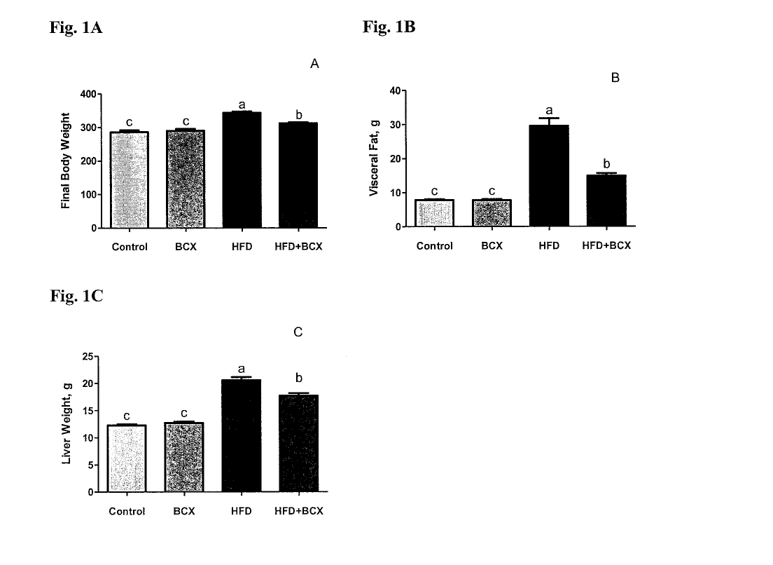

Visceral fat decreased in HFD treated BCX rats only. HFD increased visceral fat. Similarly decrease in body weight was observed in HFD treated BCX rats. Liver weight decreased in HFD treated BCX rats. (Table 1, FIG. 1A, B, C)

TABLE 1

| | Control | BC | HFD | BC |
|---|---|---|---|---|
| Initial BW (g) | 233.86 | 232.71 | 232.29 | 234.29 |
| Final BW (g) | 286.29 | 290.29 | 340.86 | 325.43 |
| Feed intake (g/d) | 23.54 | 23.88 | 19.16 | 16.87 |
| Visceral fat (g) | 7.79 | 7.75 | 29.54 | 14.93 |
| Liver (g) | 12.29 | 12.74 | 20.56 | 18.46 |

Consumption of HF diet produced a significant (P<0.001) increase in body weight (BW) compared to the consumption of normal diet (normal control group). Beta-cryptoxanthin supplementation significantly reduced the body weight as compared to the HFD control group (P<0.05). Visceral fat and liver weights were significantly higher in the HFD group as compared to the control group (P<0.05). In rats fed a HF diet supplemented with Beta-cryptoxanthin, a tendency towards a decrease in visceral fat was observed (P<0.05) (see FIG. 1).

### ii) Effect of BCX on Lipid and Lipoproteins:

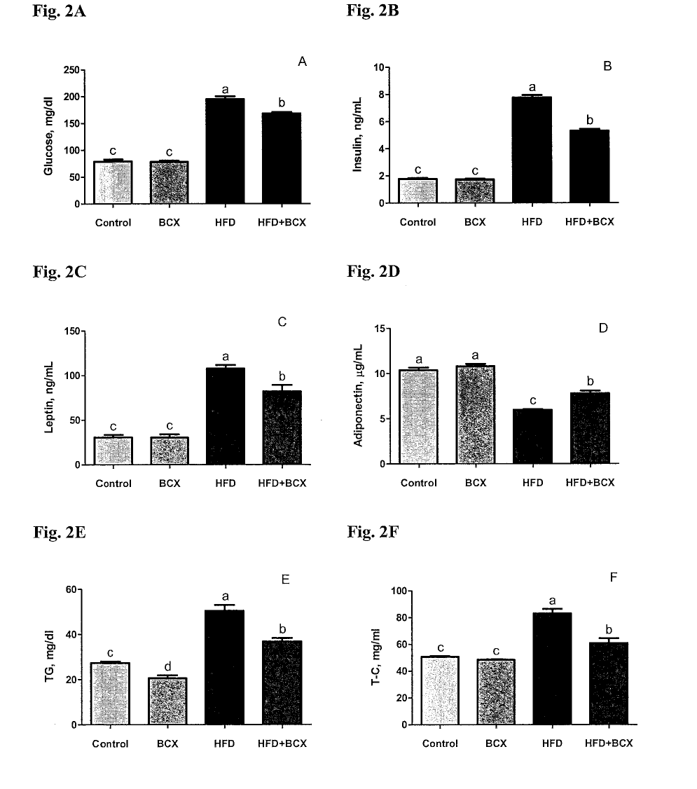

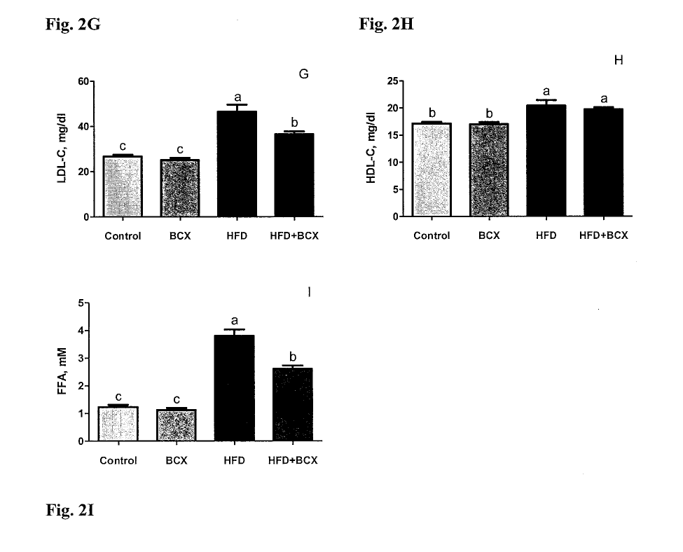

BCX reflects its significant role in cholesterol lowering effects. According to Table 2 (FIG. 2 E to H) total cholesterol decreased in HFD treated BCX rats, whereas LDL cholesterol and TG decreased in BCX treated rats. Amount of HDL, which is good cholesterol, was almost unchanged, even after administering BCX supplementation.

TABLE 2

| | ND | | HFD | |
|---|---|---|---|---|
| Details | Control | Control + BCX | HFD | HFD + BCX |
| T-C (mg/ml) | 50.80 | 48.64 | 83.14 | 60.86 |
| HDL-C (mg/dl) | 17.14 | 17.00 | 20.43 | 19.71 |
| LDL-C (mg/dl) | 26.71 | 25.00 | 46.43 | 36.43 |
| TG (mg/dl) | 27.29 | 20.57 | 50.43 | 36.70 |

iii) Effect of BCX on Metabolic Markers/Hormones:

According to the study glucose, insulin and FFA (Free Fatty Acid) level decreased in HFD treated BCX rats. On the other hand leptin was decreased significantly in HFD treated BCX and adiponectin was increased in HFD treated BCX. It was observed BCX inhibited the glucose-mediated changes in metabolic markers and lipid profile (see Table 3, FIG. 2 A-D).

TABLE 3
Serum glucose, insulin, leptin, adiponectin and lipid profile in control
and HFD rats

| | | | High Fat | |
|---|---|---|---|---|
| | Normal diet (ND) | | Diet (HFD) | |
| Item | Control | Control + BCX | HFD | HFD + BCX |
| Glucose (mg/dl) | 79.00 | 78.29 | 195.14 | 168.14 |
| Insulin (ng/mL) | 1.78 | 1.73 | 7.76 | 5.31 |
| FFA (mM) | 1.23 | 1.12 | 3.81 | 2.61 |
| Leptin (ng/mL) | 30.71 | 30.43 | 107.71 | 82.00 |
| Adiponectin (mg/mL) | 10.37 | 10.81 | 5.97 | 7.79 |

There was a significant (P<0.001) elevation in serum glucose, insulin, and leptin levels in HFD-induced obese rats compared with control rats.

Total cholesterol (T-C), free fatty acids (FFAs), triglycerides (TGs), high-density lipoprotein (HDL), low-density lipoprotein (LDL) and very-low-density lipoprotein (VLDL) were checked in serum of control and HFD-induced obese rats, respectively. The concentrations of serum lipid profiles were significantly increased in experimental obese rats as compared to the normal rats. Treatment with beta-cryptoxanthin significantly reduced the concentrations of serum glucose, insulin, leptin, lipids concentrations in obese rats but decreased adiponectin concentration in HFD rat (P<0.05) (see FIG. 2).

### iv) Effect of BCX on Oxidative Stress Markers:

Oxidative stress is significantly reduced in BCX treated rats in serum and liver. HFD rats had high thiobarbituric acid reactive substance (TBARS) and BCX treated HFD rats significantly reduced oxidative stress by reducing TBARS in retina and serum. BCX is known as provitamin A. These results further support its antioxidant activity in reducing oxidative stress markers and protects eyes and other associated conditions.

TABLE 4

| | ND | | HFD | |
|---|---|---|---|---|
| Item | Control | BC | HFD | BC |
| Serum TBARS (nmol/mL) | 0.87 | 0.67 | 1.88 | 1.64 |
| Retinal TBARS (mol/mg protein) | 80.37 | 62.67 | 228.14 | 186.43 |

b. Effect of Beta-Cryptoxanthin Compositions on Retinal Tissue in HFD Treated Rats

Beta-cryptoxanthin has several functions that are important for human health, including roles in antioxidant defense and cell-to-cell communication. Beta-cryptoxanthin is a precursor of vitamin A, which is an essential nutrient needed for eyesight, growth, development and immune response. Increase in reactive oxygen species (ROS) is one of the major retinal metabolic abnormalities associated with the development of diabetic retinopathy. NF-E2-related factor 2 (Nrf2), a redox sensitive factor, provides cellular defenses against the cytotoxic ROS. In stress conditions, Nrf2 dissociates from its cytosolic inhibitor, Kelch-like ECH-associated protein 1 (Keapl), and moves to the nucleus to regulate the transcription of antioxidant genes including the catalytic subunit of glutamylcysteine ligase (GCLC), a rate-limiting reduced glutathione (GSH) biosynthesis enzyme.

Ocular neovascularization (NV) is a major cause of the blindness associated with ischemic retinal disorders, such as proliferative diabetic retinopathy (PDR), retinopathy of prematurity (ROP), and age-related macular degeneration (AMD). Studies showed that nitric oxide (NO), produced by inducible nitric oxide synthase (iNOS), plays an important role in eye diseases such as glaucoma, ROP and AMD.

Animals fed on HFD showed an increased upregulation of inflammatory and proangiogenic markers. This animal model may be useful to study mechanisms of diabetic retinopathy and therapeutic targets.

According to FIG. 5 the intensity of the bands was quantified by densitometric analysis. Data are expressed as a ratio of normal control value (set to 100%). The bar represents the standard error of the mean. Blots were repeated at least 3 times (n=3) and a representative blot is shown. β-actin was included to ensure equal protein loading.

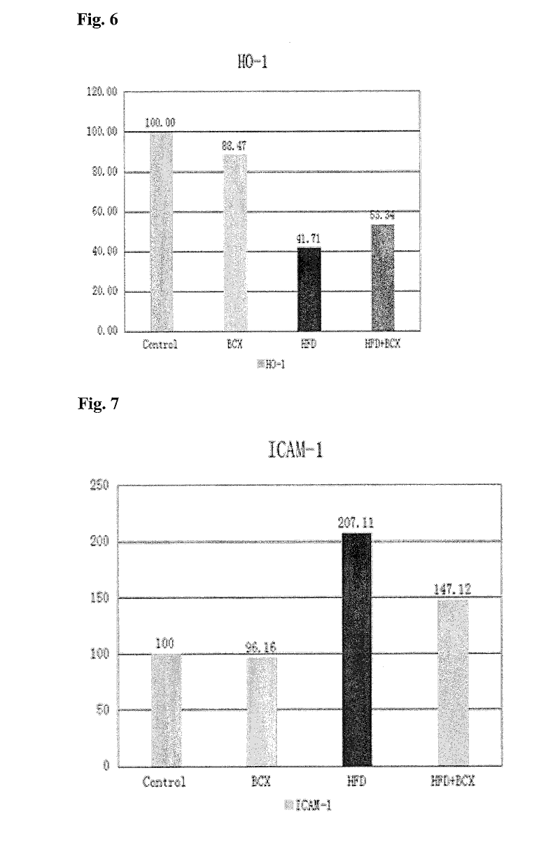

HO-1 is a sensitive marker for assessing light-induced insult in the retina. Increased expression of HO-1 is thought to be a cellular defense against oxidative damage, and its expression may play an important role in protecting the retina against light damage (see FIG. 6).

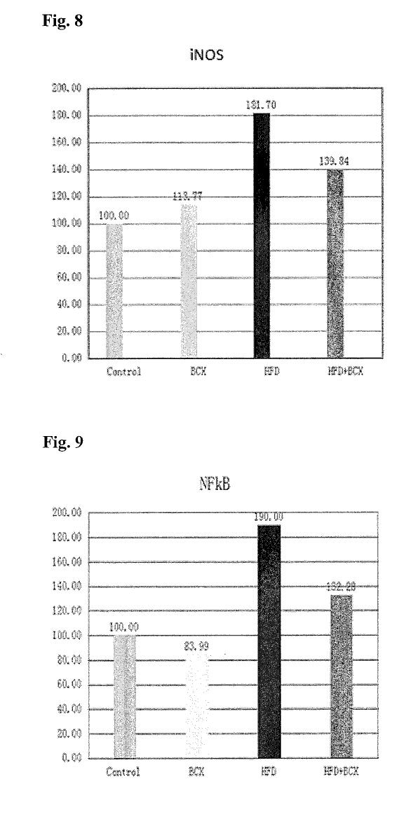

Leukocytes play a critical role in ocular diseases such as uveitis, diabetic retinopathy, and choroidal neovascularization. Intercellular adhesion molecule (ICAM)-1 is essential for the migration of leukocytes. Control of ICAM-1 expression may lead to therapies for these diseases. Down regulation of ICAM-1 expression to reduce retinal neovascular disease by inhibiting leukocyte infiltration (see FIG. 7).

To treat/prevent eye diseases like AMD, the effect of BCX on inhibition or induction of iNOS can be checked in animal models. BCX decreased iNOS and may be potential for neovascualarization (see FIG. 8).

It is also observed that BCX inhibited the glucose-mediated induction of NF-kB expression in retina (see FIG. 9) which suggest that selective inhibition of the NFkB pathway in glial can be potent clinical approach for the treatment of vision loss in glaucoma.

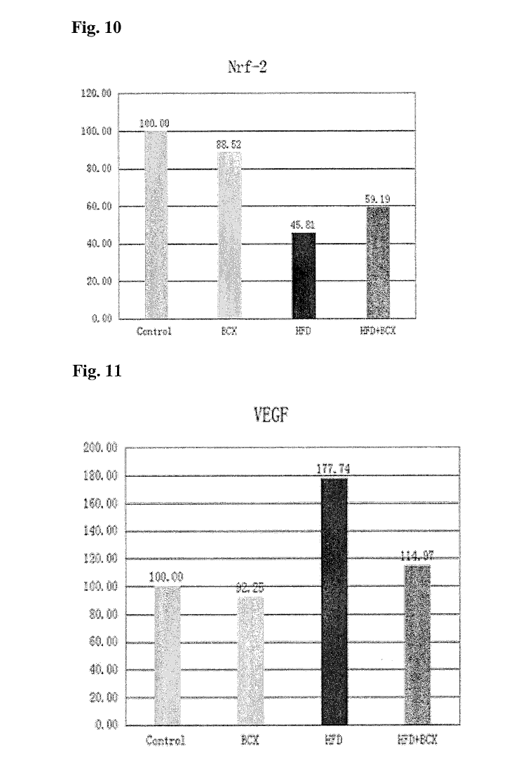

Nrf2 is involved in the cytoprotective mechanism in the retina in response to ischemia-reperfusion injury and suggests that pharmacologic induction of Nrf2 could be a new therapeutic strategy for retinal ischemia-reperfusion and other retinal diseases (see FIG. 10).

VEGF has been considered to be a mediator of diabetic retinopathy. Inhibition of VEGF reduces retinal neovascularization (see FIG. 11).

c. Effect of Beta-Cryptoxanthin Compositions on Liver Tissue in HFD Treated Rats

### Animals and Diets

The experiment was performed using 28 male Sprague-Dawley rats (8 weeks old, weighing 180±20 g), purchased from the Inonu University Laboratory Animal Research Center (Malatya, Turkey). Rats were housed in cages in a temperature and humidity controlled environment, on a 12-hr light and 12-hr dark cycle, designed for the purpose of the study. The temperature inside the rat cages was 21±2° C., relative humidity was 55±5% and consecutive light-dark cycles lasted 12 hours. The protocol of the study was approved by the Animal Experimentation Ethics Committee of Inonu University (Malatya, Turkey). All procedures involving rats were conducted in strict compliance with relevant laws, the Animal Welfare Act, Public Health Services Policy, and guidelines established by the Institutional Animal Care and Use Committee of the Institute. Prior the starting the experiment, animals were assigned to either a regular diet (control; 12% of calories from fat) or a high-fat diet (HFD, 42% of calories from fat). Control or HFD composed according to the American Institute of Nutrition AIN-93 (Reeves et al., 1993) recommendations of casein (20%), soybean oil (7%), wheat starch (53.2%), sucrose (10%), potato starch (5%), 1-cysteine (0.3%), vitamin mix AIN-93M (1%) and mineral mix AIN-93M (3.5%). The high-fat diets (42% calories from fat) were obtained from the basal AIN-93 diet, by replacement of wheat starch with fat (tallow 15% and soybean oil 10%). For induction of obesity (insulin resistance), the rats were fed with HFD for 12 weeks

### Experimental Protocol

After a one week period of adaptation to laboratory conditions, the animals were randomly divided into four equal groups: 1) Control (n=7): untreated rats were allowed free access to a standard diet; 2) BCX Group, rats were fed a standard diet supplemented with beta-cryptoxanthin (n=7) 2.5 mg/kg; 3) HFD Group (high-fat diet; 42% of calories from fat; n=7): rats were fed a high-fat diet 4) HFD+BCX Group, rats were fed a high-fat diet (42% of calories from fat; n=7) supplemented with beta-cryptoxanthin 2.5 mg/kg. 5% suspension of beta-cryptoxanthin was dissolved in corn oil. At the end of the experiment, all rats were sacrificed by cervical dislocation. Blood samples were taken from rats in the morning upon overnight fasting for biochemical analyses and their visceral fat and liver samples were removed and weighed after sacrificing the animals.

### Laboratory Measurements

Blood was collected by cardiac puncture using an anticoagulant-free vacutainer tube, later centrifuged at 3,000×g for 10 min to obtain serum and kept frozen at −80° C. until it was assayed for biochemical parameters and malondialdehyde (MDA). Serum biochemical parameters were estimated using an automatic analyzer (Samsung LABGEO PT10, Samsung Electronics Co, Suwon, Korea). Repeatability and device/method precision of LABGEO^{PT10} was established according to the IVR-PT06 guideline. Serum insulin, leptin and adiponectin levels were measured with the Rat Insulin Kit (Linco Research Inc, St. Charles, Mo., USA) by ELISA (Elx-800; Bio-Tek Instruments Inc, Vermont, USA).

The total antioxidant capacity (TAC) was measured using an antioxidant assay kit (Sigma, St Louis, Mo., USA). Trolox was used as an antioxidant standard to calculate Trolox equivalent antioxidant capacity; absorbance readings were taken at 520 nm. Lipid peroxidation was measured in terms of MDA formation, which is the major product of membrane lipid peroxidation done by a previously described method (Karatepe, 2004) with slight modification.

The liver MDA content was measured by high performance liquid chromatography (HPLC, Shimadzu, Tokyo, Japan) using a Shimadzu UV-vis SPD-10 AVP detector and a CTO-10 AS VP column in a mobile phase consisting of 30 mM KH2PO4 and methanol (82.5+17.5, v/v; pH 3.6) at a flow rate of 1.2 ml/min. Column effluents were monitored at 250 nm and the volume was 20 μl. The liver homogenate (10%, w/v) was prepared in 10 mM phosphate buffer (pH 7.4), centrifuged at 13,000×g for 10 min at 4° C., and the supernatant was collected and stored at −80° C. for MDA analysis (Karatepe, 2004). Activities of superoxide dismutase (SOD), catalase (CAT) and glutathione peroxidase (GPx) of homogenized liver were measured using a commercial kit (Cayman Chemical, Ann Arbor, Mich., USA) according to the manufacturer's instructions.

### Western Blots Analysis

For Western blot analyses protein extraction was performed by homogenizing the liver in 1 ml ice-cold hypotonic buffer A, containing 10 mM2-[4-(2-Hydroxyethyl)-1-piperazinyl]ethanesulfonic acid [HEPES] (pH 7.8), 10 mMKC1, 2 mM MgCl2, 1 mM DTT, 0.1 mM EDTA, and 0.1 mMphenylmethylsulfonyl-fluoride (PMSF). The homogenates were added with 80 μl of 10% Nonidet P-40 (NP-40) solution and then centrifuged at 14,000×g for 2 min. The precipitates were washed once with 500 μl of buffer A plus 40 μl of 10% NP-40, centrifuged, re-suspended in 200 μl of buffer C [50 mM HEPES [pH 7.8], 50 mMKC1, 300 mM NaCl, 0.1 mM EDTA, 1 mM dihiothreitol [DTT], 0.1 mM PMSF, 20% glycerol], and recentrifugedat 14,800×g for 5 min. The supernatants were collected for determinations of NF-κB, VEGF, iNOS, ICAM, Nrf2, and HO-1 according to the method described by Sahin et al. [2012]. Equal amounts of protein (50 μg) were electrophoresed and subsequently transferred onto a nitrocellulose membrane (Schleicher and Schuell Inc., Keene, N.H., USA).

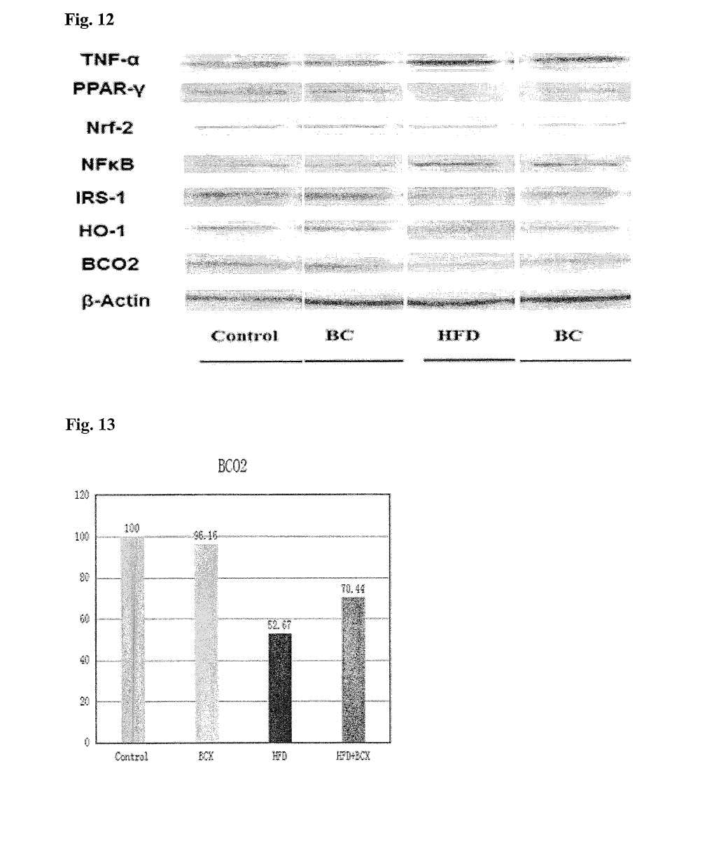

Antibodies against NF-κB, TNF-α, Nrf2, HO-1, PPAR-γ, and p-IRS1, (Abcam (Cambridge, UK) were diluted (1:1000) in the same buffer containing 0.05% Tween-20. Protein loading was controlled sing a monoclonal mouse antibody against β-actin (A5316; Sigma). Bands were analyzed densitometrically using an image analysis system (Image J; National Institute of Health, Bethesda, USA). (FIG. 12)

### Statistical Analysis

Sample size was calculated based on a power of 85% and a P-value of 0.05. Data are expressed as mean±standard deviation. Differences among the groups were evaluated using the General Linear Model (GLM) procedure of SAS at baseline. If ANOVA indicated significance, a Fisher's multiple comparison test was performed. The alpha level of significance was set at P<0.05.

### Effect of Beta-Cryptoxanthin Compositions on Oxidative Metabolites and Antioxidant Capacity in Liver Tissue

Antioxidant capacity, catalase, superoxide dismutase (SOD) increased in BCX treated rats and decreased TBARS in liver. These results suggest BCX protects liver from oxidative stress due to hyperglycemia.

TABLE 5

| | ND | | HFD | |
|---|---|---|---|---|
| Details | Control | BC | HFD | BC |
| Liver TBARS (nmol/mg protein) | 1.82 | 1.56 | 3.82 | 2.98 |
| Serum TAC (U/mL) | 1.18 | 1.19 | 0.46 | 0.64 |
| Liver SOD (U/mg protein) | 182.86 | 198.57 | 106.29 | 130.43 |
| Liver CAT | 299.71 | 306.14 | 253.43 | 271.57 |
| (U/mg protein) | | | | |
| Liver GSHPx | 41.29 | 50.29 | 16.86 | 17.43 |
| (U/mg protein) | | | | |

GSHPx: (Glutathione peroxidase);
CAT: (catalase);
SOD: (superoxide dismutase);
TAC: (total antioxidant capacity)

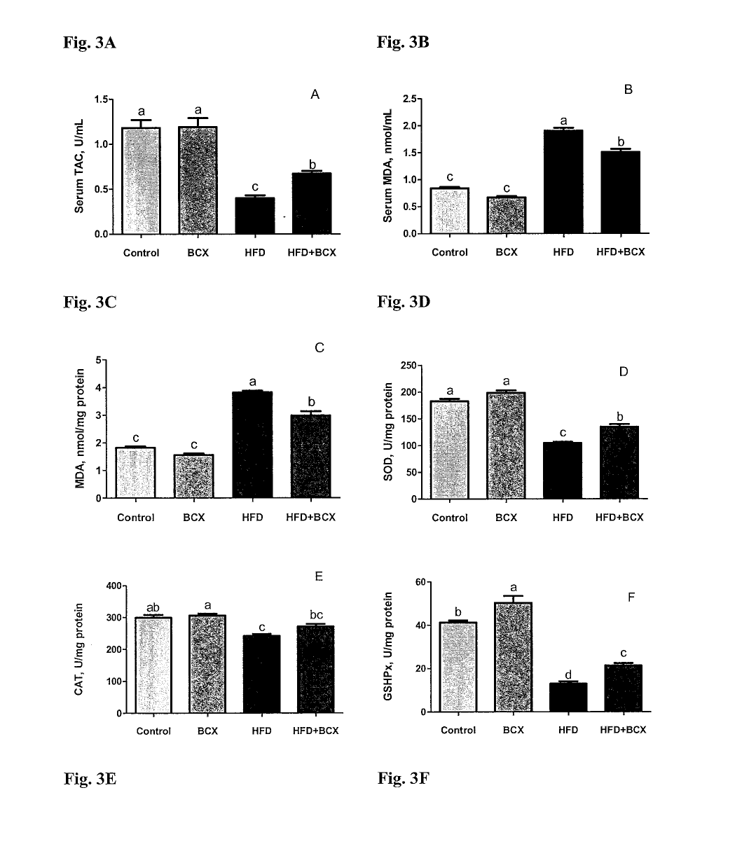

Rats fed a HFD had lower levels of total antioxidant capacity (TAC) activities of superoxide dismutase (SOD), catalase (CAT) and glutathione peroxidase (GSH-Px) and higher malondialdehyde (MDA) concentration than rats fed a standard diet (P<0.001 for all). Beta-cryptoxanthin administration increased activities of these enzymes and decreased MDA concentration in rats fed a HFD (P<0.05) (See FIG. 3)

BCX compositions upregulate BCO2 expression. BCO2 acts as a protective antioxidant and plays a crucial role in protection against oxidative damage (see FIG. 13).

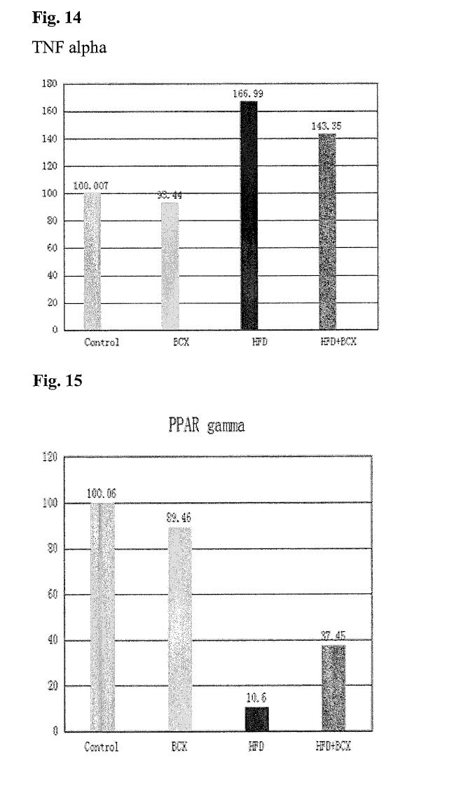

It is also observed that decrease in TNF alpha decreases oxidative stress in liver tissue (see FIG. 14).

BCX activated PPAR gamma in HFD treated rats shows that BCX had a significant role in CMS and antioxidant pathways (see Figure: 15).

BCX compositions decrease Nrf2 expression, which improves glucose homeostasis, possibly through its effects on fibroblast growth factor 21 (Fgf 21) and/or insulin signaling in liver tissue of HFD rats (see FIG. 16).

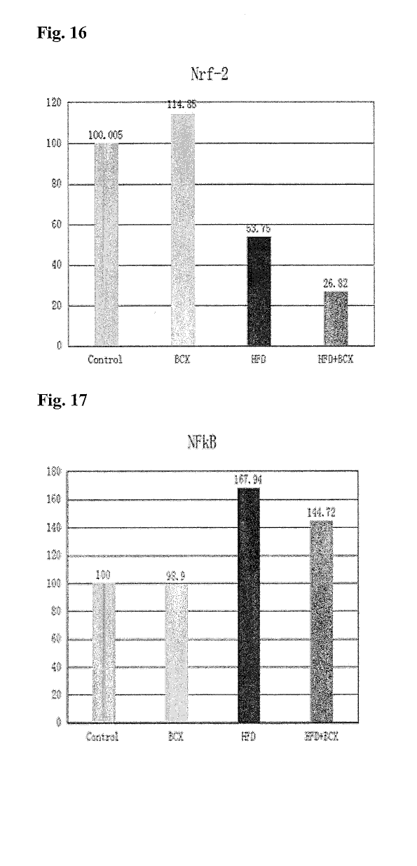

Further NFkB decreased in HFD treated with BCX. These results show BCX as antioxidant anti-inflammatory provitamin A (see FIG. 17).

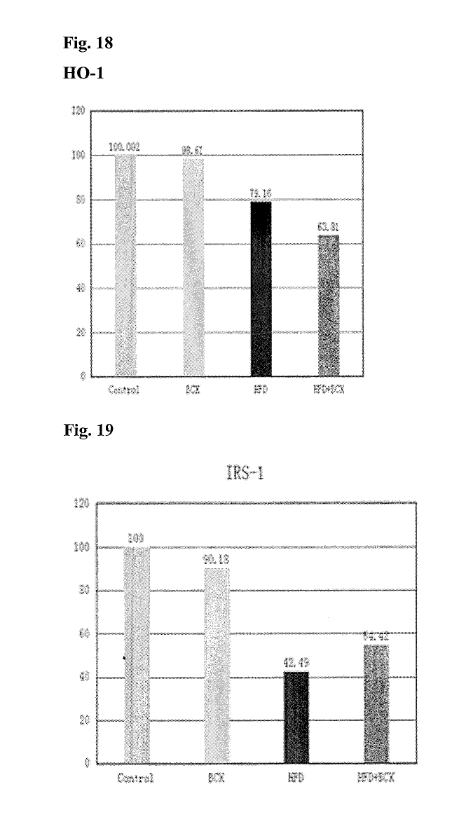

According to FIG. 18 increased HO-1 is associated with diabetes and may contribute to the progression of insulin resistance in obese patients by promoting chronic inflammation. HFD rats treated with BCX decreased HO1.

Decreased IRS-1 was also associated with a decrease in glucokinase expression and a trend toward increased blood glucose. HFD rats treated BCX increased IRS-1 in liver so there is a potential decrease in blood glucose and glucose management (See FIG. 19).

It is observed that beta-cryptoxanthin inhibited liver NFkB and TNF-α expression by 22% and 14% and enhanced liver Nrf2, HO-1, PPAR-α, and p-IRS1 levels were enhanced by 1.1.43, 1.41, 3.53, and 1.33, fold, respectively (P<0.001).

We claim:

1. A method for prevention and treatment of cardiometabolic syndrome, comprising administering a beta-cryptoxanthin composition in an effective amount to a subject fed with high fat diet; and evaluating effect of composition on cardiometabolic markers, oxidative stress markers and inflammatory makers, to assess overall management of cardiometabolic syndrome.

2. The method of claim 1, wherein the beta-cryptoxanthin composition is administered to the subject fed with high fat diet in a daily dose of about 0.1 to 100 mg/kg body weight and evaluated to assess overall management of cardiometabolic syndrome.

3. The method of claim 1, wherein the beta-cryptoxanthin composition is administered in an effective amount to the subject fed with high fat diet and is evaluated on cardiometabolic markers including one or more of body weight, body fat, lipid profile, blood glucose, blood insulin, leptin, and liver weight.

4. The method of claim 3, wherein the lipid profile is evaluated through assessment of one or more of triglycerides levels, serum LDL levels, serum HDL levels and total cholesterol levels, when administered to the subject fed with high fat diet.

5. The method of claim 1, wherein the beta-cryptoxanthin composition is administered in an effective amount to the subject fed with high fat diet and is evaluated on oxidative stress markers and inflammatory markers related to vital body organs such as eye and liver.

6. The method of claim 4, wherein the evaluating includes checking an effect on oxidative stress markers including one or more of SOD, CAT, GPx, and TBARS to assess liver function and indications of cardiometabolic syndrome.

7. The method of claim 4, wherein the evaluating includes checking effect on inflammatory stress markers including one or more of HO-1, ICAM, INOS, NfkB, NrF2, and VEGF to assess eye function and indications of cardiometabolic syndrome.
